(19) United States
(12) Patent Application Publication
(10) Pub. No.: US 2005/0153449 A1
(43) Pub. Date: Jul. 14, 2005

(54) Method for producing insulin-secreting stem cells and cells derived therefrom

(76) Inventors: Steven C. Shanks, Mesa, AZ (US); Susan M.L. Lim, Singapore (SG); Kevin Slattery, Chandler, AZ (US)

Correspondence Address: ETHERTON LAW GROUP, LLC, 5555 E. VAN BUREN STREET, SUITE 100, PHOENIX, AZ, 85008, US

(21) Appl. No.: 11/036,625

(22) Filed: Jan. 13, 2005

Related U.S. Application Data
(60) Provisional application No. 60/536,407, filed on Jan. 13, 2004.

(51) Int. Cl. C12N 15/01; C12N 5/08

(52) U.S. Cl. 435/446; 435/366

(57) ABSTRACT

The present invention is a method for producing an insulin-secreting pre-adipocyte stem cell and the cells derived therefrom. The preferred method comprises obtaining fat cells from a human; treating the fat cells with low-level laser energy; culturing the treated fat cells and suspending them in media together with an insulin gene plasmid; and subjecting the fat cells to transfection. The transfection is facilitated with the application of low-level laser energy.

### CROSS-REFERENCE TO RELATED APPLICATIONS

This application claims the benefit of co-pending U.S. Provisional Application No. 60/536,407 filed Jan. 13, 2004.

### FIELD OF INVENTION

This invention relates generally to a method for producing insulin-secreting cells. This invention relates more particularly to producing such cells from stem cells via transfection facilitated by laser energy.

### BACKGROUND

A stem cell is an undifferentiated cell found in a differentiated tissue that can renew itself and (with certain limitations) differentiate to yield all the specialized cell types of the tissue from which it originated. Stem cells have two important characteristics that distinguish them from other types of cells. First, they are unspecialized cells that renew themselves for long periods through cell division. The second is that under certain physiologic or experimental conditions, they can be induced to become cells with special functions such as the beating cells of the heart muscle or the insulin-producing cells of the pancreas. In this way it has been hypothesized by scientists that stem cells may, at some point in the future, become the basis for treating diseases such as Parkinson's disease, diabetes, and heart disease as the healthy cells are transplanted to a diseased pateint to replace damaged cells.

Scientists primarily work with two kinds of stem cells in humans: embryonic stem cells and adult stem cells. Human embryonic stem cells are obtained from human embryos, and are thus currently the subject of ethical and political considerations that make them problematic to work with. Adult stem cells, however, avoid such problems and are thus more desirable to work with. Adult stem cells may suffer their own problems, however. Stem cells cultured from one patient may be rejected by another patient, and despite requiring immunosuppressants, ultimately may still be rejected. It would be desirable to transplant autologous cells from a patient, obviating the need for immunosuppression.

Until recently, it had been thought that adult stem cells typically generate the cell types of the tissue in which they reside. A blood-forming adult stem cell in the bone marrow, for example, normally gives rise to the many types of blood cells such as red blood cells, white blood cells and platelets. A blood-forming cell in the bone marrow could not give rise to the cells of a very different tissue, such as nerve cells in the brain. However, a number of experiments over the last several years have raised the possibility that stem cells from one tissue may be able to give rise to cell types of a completely different tissue. Examples include blood cells becoming neurons, liver cells that can be made to produce insulin, and hematopoietic stem cells that can develop into heart muscle. Therefore, exploring the possibility of using adult stem cells for cell-based therapies has become a very active area of investigation by researchers.

There are several known methods for transfection to stimulate stem cell differentiation. For example, viral vectors can efficiently transfer genes of interest to a broad range of mammalian cell types. However, despite being derived from non-pathogenic viruses, the potential for pathogenicity and immmunorejection makes such the use of viral vectors somewhat controversial. Another method involves electroporation which is a well-known technique by which large molecules are inserted into cells under a large electric field. It has typically been performed on many cells at once, but low success rates and high cell mortality have made the technique less suited for single cells and stem cells that are typically available in very small quantities. Chemical transfection suffers from detrimental effects of the chemical environment used to insert the gene. Physical transfection can be accomplished with microinjection using a microcapillary pipette. It would be desirable to develop a differentiation method that avoids issues associated with these methods.

An article recently published in Tissue Engineering showed that adipocytes or fat cells taken from liposuction procedures can be utilized as an excellent source of stem cells. Researchers first take the fat and fluid drained from the hips, buttock and stomachs of liposuction patients. The material, referred to as lipoaspirate, is then washed, purified and treated with an enzyme to break down the matrix holding the cells together and compared to stem cells from bone marrow. Scientists found that a half-pound of the fatty substance yielded as many as 50 million to 100 million undifferentiated stem-like cells.

Dr. Rodrigo Neira, a plastic surgeon from Cali, Colombia developed a technique that prepares the fat for easier extraction during liposuction procedures. In his paper, “Fat Liquefaction: Effect from low level Laser Energy on Adipose Tissue”, he uses a series of MRI's and SEM's to document how the Erchonia® 635 nm low level laser creates a “transient pore” in the cell membrane of the adipocyte following 4-6 minutes of irradiation. Sufficient laser energy is applied to the adipocyte or fat cell to release at least a portion of the intracellular fat through the “transient pore” and into the extra-cellular space, thereby facilitating extraction. U.S. Pat. No. 6,605,079, issued to Dr. Niera and one of the inventors of the present invention, describes the method in more detail. That patent is incorporated herein by reference.

Subsequent cell cultures were performed on these extracted fat cells which confirmed 100% viability following laser irradiation. This is a significant finding since the current use of ultrasonic liposuction destroys the adipocyte rendering it useless for harvesting or transplanting. The fact that the cellular structure of the adipocyte was manipulated by the laser without any damage is even more significant.

Low-level lasers, such as those described in U.S. Pat. Nos. 6,013,096 and 6,746,473, incorporated herein by reference, are used in the treatment of a broad range of conditions. Low level laser therapy (LLLT) improves wound healing, reduces edema, and relieves pain of various etiologies, including successful application post-operatively to liposuction to reduce inflammation and pain. LLLT is also used during liposuction procedures to facilitate removal of fat by causing intracellular fat to be released into the interstice, as described by Neira. It is also used in the treatment and repair of injured muscles and tendons.

LLLT utilizes low level laser energy, that is, the treatment has a dose rate that causes no immediate detectable temperature rise of the treated tissue and no macroscopically visible changes in tissue structure. Consequently, the treated and surrounding tissue is not heated and is not damaged. There are a number of variables in laser therapy including the wavelength of the laser beam, the area impinged by the laser beam, laser energy, pulse frequency, treatment duration and tissue characteristics. Norbert Wiener in Energy Medicine, The Scientific Basis, coined the term “cybernetics” which is defined as the science of communication and control. The underlying theory advocates that a small energy field applied at the appropriate place and time can shift the course of an organism. This has become a productive area of research, for all living processes are ultimately carried out by cells and by the molecules and by the energy fields they produce. Scientists are now learning precisely which steps in the cellular/molecular/electromagnetic cascade are particularly sensitive to exogenous energy fields and which ones are not. They are also discovering how minute signals from the environment are amplified to produce large cellular effects. The significance of cellular amplification was recognized by the 1994 Nobel Prize in Physiology or Medicine for the discovery of G-proteins and the role of these proteins in signal transduction in cells.

It is thought that receptors on the cell surface are the primary sites of action of low frequency electromagnetic fields. It is at the receptor site that cellular responses are triggered by hormones, growth factors, neurotransmitters, light and other electromagnetic signals. Membrane proteins closely associated with receptors, such as adenylate cyclases and G proteins, couple a single molecular event at the cell surface to the influx of a huge number of calcium ions. These calcium ions enter the cell and activate a variety of enzyme molecules. These enzymes in turn act as catalysts and greatly accelerate the biochemical processes. The frequency of the stimulus is crucial. Separate studies of lymphocytes stimulated with a mitogen showed that a weak 3 Hz pulsed magnetic field sharply reduced calcium influx, while a 60 Hz signal, under identical conditions increased calcium influx. New research is revealing how free radicals, including nitric oxide, are involved in the coupling of electromagnetic fields to chemical events in the signal cascade. Again, the medical importance of this research has been recognized by 1998 Nobel Prize in Physiology or Medicine for the discoveries concerning nitric oxide as a signaling molecule in the cardiovascular system.

The human body derives specialized cells from stem cells by specific differentiation signals. For example, the tanning response of skin cells to the sun shows that biochemical responses are created in cells by applying ultraviolet electromagnetic radiation, whereas the cells may have little or no response to visible light. This indicates that a given radiation wavelength or frequency—or range of frequencies—generates a specific response in each cell. It is logical to reason, then, that cell lineages (cartilage, nerve, bone, etc) react to a specific wavelength or frequency to achieve a specific outcome. That is, cell lineages would be frequency specific for a desired response. Therefore, it is theorized that totipotential stem cells can be treated or irradiated with the specific wavelength or frequency and coaxed or manipulated into differentiating into a predetermined cell line. It would be desirable to apply energy of a specific wavelength and frequency to produce preadipocyte stem cells.

Therefore, an object of this invention is to provide a method for the treatment of diabetes. It is another object to provide an insulin-secreting stem cell. It is a further object to provide an insulin-secreting stem cell that is autologous with the diabetic patient being treated. It is another object to provide a method of creating insulin-secreting stem cells from fat tissue or lipoaspirate. It is another object to provide a pre-adipocyte stem cell using low level laser energy. It is another object to facilitate the transfection of the insulin gene into the stem cells with the use of low level laser energy.

### SUMMARY OF THE INVENTION

The present invention is a method for producing an insulin-secreting pre-adipocyte stem cell and the cells derived therefrom. The preferred method comprises obtaining fat cells from a patient; treating the fat cells with low-level laser energy; culturing the treated fat cells and suspending them in media together with an insulin gene plasmid; and subjecting the fat cells to transfection. The transfection is facilitated with the application of low-level laser energy.

### DETAILED DESCRIPTION OF THE INVENTION

Fat tissue is obtained from human patients, typically in a minor surgical procedure. Preferably the fat tissue is obtained as lipoaspirate resultant from liposuction. To avoid adverse immune systems responses, it is preferable to obtain lipoaspirate from the patient who is to be treated with the stem cell therapy, thereby avoiding the need for immunosuppression. In the preferred embodiment, the patient is a diabetic who will be treated with the transplantation of autologous healthy insulin-producing cells.

In the preferred embodiment, the fat tissue undergoes treatment ex-vivo with a low level laser for a defined short period of time. Preferably the treatment does not exceed 20 minutes and, in an alternate embodiment, is not applied at this point in the method (treatment time is zero). The exposure to low level laser energy opens the pores in the adipocyte cell membrane and facilitates the leakage of lipid out of the fat cells. This lipid, together with other impurities, is discarded.

Preferably the applied laser energy has a wavelength of specified wavelength between 630-640 nm. Laser energy sources are known in the art for use in low-level laser therapy. They include Helium-Neon lasers having a 632 nm wavelength and semiconductor diode lasers with a broad range of wavelengths between 600-800 nm. The laser energy sources in the preferred embodiment are semiconductor laser diodes that produce light in the red range of the visible spectrum, having wavelengths of about 635 nm. Solid state and tunable semiconductor laser diodes may also be employed to achieve the desired wavelength.

Different therapy regimens require diodes of different wattages and pulse frequencies. The preferred laser diodes are of less than one watt. For ease of reference, pulse frequency can be referred to in shorthand notation in pulses/second, or Hz. Pulse frequency from 0 to 100,000 Hz may be employed to achieve the desired effect on the patient's tissue. At 100,000 Hz, the pulse frequency is 0.00001 second. At 0 Hz, a continuous beam of laser light is generated. The goal of the LLLT regimen is to deliver laser energy to the targeted cells utilizing a pulse frequency short enough to sufficiently energize the targeted cell and avoid thermal damage to it.

The resultant fat cells are clumped in loose aggregates referred to as cell pellets through centrifugation. The cell pellets are then resuspended in media and plated at about 4×10(4) cells/cm² in culture dishes. The cells are washed with isotonic fluids and cultured in media, including a serum which is conducive to cell attachment and growth. Preferably the serum is autologous to the patient. The cells are cultured in an incubator for a defined period of time and temperature. Preferably the incubation is at 37C for not more than four weeks.

The cells are then subjected to transfection to enable the insulin gene to enter the cell. Preferably the gene is carried on a plasmid. The cultured pre-adipocyte stem cells are suspended in media together with the insulin gene plasmid at a predetermined concentration, preferably of between 0.5 to 2 micrograms per 1.6 million cells. Brief pulses of energy are delivered to the cells, preferably low level laser energy, which creates transient pores in the cell membrane to facilitate the entry of the insulin gene. Any transfection method may be used, but the use of low level lasers offers several advantages over conventional gene transfer techniques such as electroporation, viral vectors, CaCl₂ or PEG mediated transformation. These advantages include speed, ease of operation and higher transformation efficiencies, and avoidance of detrimental effects of chemicals (PEG), or viruses. Cessation of laser treatment effectively removes the external field (laser) and permits the resealing of the cell membrane electropores, thereby ensuring the survival of the laser-treated cells.

Numerous factors affect the transfection yield, and each must be empirically optimized. However, it has been determined that the best transfection yield is achieved when the following parameters have been optimized: cellular state of the cells; physiochemical conditions (pH and temperature); insulin DNA concentration; and laser criteria, particularly wattage, pulse frequency, wavelength, and duration of treatment.

The preferred ranges for transfection are cells plated at 20-60% confluency, 24-48 hours before transfection. The culture is at a pH of at least 6 at a temperature of 37C. The insulin DNA concentration is 0.5 to 2 micrograms per 1.6 million cells. The laser emits a wavelength between 600 to 640 nm, and is applied to the cells in short repetitive pulses over 5 to 30 minutes. More preferably the laser is a 635 nm semiconductor laser diode of 5 milliwatts, applied at 1000 Hz over 5 minutes.

The successful transfection of the preadipocyte stem cells with the insulin gene is documented on western blot analysis, immunofluorescent and other studies. The viability of the transfected cells and measurement of secreted insulin is then assessed. This is performed by assaying cell culture supernatants of transfected cells for insulin. The gene-engineered preadipocyte stem cells may then be selected for clinical trials, during which they may be injected into diabetic patients as autografts.

While there has been illustrated and described what is at present considered to be a preferred embodiment of the present invention, it will be understood by those skilled in the art that various changes and modifications may be made, and equivalents may be substituted for elements thereof without departing from the true scope of the invention. Therefore, it is intended that this invention not be limited to the particular embodiment disclosed as the best mode contemplated for carrying out the invention, but that the invention will include all embodiments falling within the scope of the appended claims.

1. A method for producing an insulin-secreting stem cell, the method comprising: suspending one or more fat cells in media together with an insulin gene and treating the suspended fat cells with laser energy.

2. The method according to claim 1 wherein the fat cells are from fat tissue or lipoaspirate.

3. The method according to claim 1 wherein the laser energy is applied for not more than 20 minutes.

4. The method according to claim 1 wherein the insulin gene is supplied a predetermined concentration.

5. The method according to claim 4 in which the insulin DNA concentration is 0.5 to 2 micrograms per 1.6 million cells.

6. The method of claim 1 wherein the insulin gene is carried by a plasmid.

7. The method according to claim 1 wherein the laser energy is less than 1 watt.

8. The method according to claim 1 wherein the laser energy is in the visible spectrum.

9. The method according to claim 1 wherein the laser energy is in the red range of the visible spectrum.

10. The method according to claim 1 wherein the wavelength of the laser energy is about 635 nm.

11. The method according to claim 1 wherein the pulse frequency of the laser energy is less than 100,000 Hz.

12. The method of claim 1 which further comprises:
obtaining the fat cells from a human; and
culturing the fat cells.

13. The method according to claim 12 wherein the media includes a serum autologous to the human.

14. The method according to claim 12 wherein culturing further comprises treating the fat cells in culture with laser energy.

15. The method according to claim 12 wherein the culturing occurs in an incubator.

16. The method according to claim 15 wherein the incubation is for not more than four weeks.

17. The method according to claim 12 wherein the laser energy applied to the fat cells in culture is less than 1 watt.

18. The method according to claim 14 wherein the laser energy applied to the fat cells in culture is in the visible spectrum.

19. The method according to claim 14 wherein the laser energy applied to the fat cells in culture is in the red range of the visible spectrum.

20. The method according to claim 13 wherein the laser energy applied to the fat cells in culture is about 635 nm.

21. The method according to claim 13 wherein the laser energy applied to the fat cells in culture is less than 100,000 Hz.

22. A method for producing an insulin-secreting pre-adipocyte stem cell, the method comprising:
a. obtaining fat cells from lipoaspirate of a diabetic human;
b. exposing the fat cells with a first laser energy treatment to facilitate the removal of lipid out of the fat cells to create laser-treated cells;
c. culturing the laser-treated cells by washing them with isotonic fluids and placing them in media that includes an serum autologous to the human in an incubator for a defined period of time not exceeding 4 weeks to create cultured cells;
d. suspending the cultured cells in media together with an insulin gene plasmid to create suspended cells;
e. treating the suspended cells with a second low-level laser energy to facilitate the transfection of the insulin gene.

23. The method according to claim 22 wherein the first and second laser energy treatments use laser sources of less than 1 watt.

24. The method according to claim 22 wherein the first and second laser energy treatments use laser energy in the visible spectrum.

25. The method according to claim 22 wherein first and second laser energy treatments use laser energy in the red range of the visible spectrum.

26. The method according to claim 22 wherein the wavelength of first laser energy treatment is about 635 nm.

27. The method according to claim 22 wherein the first laser energy treatment uses a pulse frequency of less than 100,000 Hz.

28. An insulin-secreting pre-adipocyte stem cell.

29. The cell of claim 28 produced by the method of claim 1.

30. The cell of claim 28 produced by the method of claim 22.

31. An insulin-secreting stem cell which has been differentiated from stem cells found in fat tissue.

32. An insulin-secreting stem cell which has been differentiated from stem cells found in lipoaspirate.

33. A method of treating disease in a human which comprises administering cells derived from the method of claim 1.

34. A method of claim 33 in which the human being treated is the same as the human from which the fat cells are obtained.

35. A method of treating disease in a human which comprises administering cells derived from the method of claim 22.

36. A method of claim 35 in which the human being treated is the same as the human from which the fat cells are obtained.

37. A method of treating disease in a human which comprises administering cells of claim 28.

38. A method of treating disease in a human which comprises administering cells of claim 31.

39. A method of claim 38 in which the human being treated is the same as the human from which the fat tissue is obtained.

40. A method of treating disease in a human which comprises administering cells of claim 32.

41. A method of claim 40 in which the human being treated is the same as the human from which the lipoaspirate is obtained.
